(19) United States
(12) Patent Application Publication
(10) Pub. No.: US 2025/0134914 A1
(43) Pub. Date: May 1, 2025

(54) GLUCOSE TOLERANCE BEVERAGES AND METHODS OF USE

(71) Applicant: Jacqueline Berg, Los Angeles, CA (US)

(72) Inventor: Jacqueline Berg, Los Angeles, CA (US)

(21) Appl. No.: 18/682,906

(22) PCT Filed: Sep. 21, 2022
(86) PCT No.: PCT/US2022/076759

§ 371 Date: Feb. 10, 2024

Related U.S. Application Data
(60) Provisional application No. 63/233,025, filed on Aug. 13, 2021.

(51) Int. Cl. A61K 31/7004; A23L 2/38; A23L 2/39; A23L 2/52; A61K 9/00; A61K 36/47; A61K 36/534; A61K 36/752; A61K 36/899; A61K 49/00

(52) U.S. Cl. CPC A61K 31/7004; A23L 2/38; A23L 2/39; A23L 2/52; A61K 9/0095; A61K 36/47; A61K 36/534; A61K 36/752; A61K 36/899; A61K 49/0004

(57) ABSTRACT

Aspects described herein relate to a glucose tolerance beverage composition comprising, consisting of, or consisting essentially of: a) 50 g, 75 g, or 100 g dextrose from a non-GMO source; b) organic peppermint leaf; and c) organic or crystalized lemon; wherein the beverage composition is free of or substantially free of protein or DNA from GMOs, Brominated Vegetable Oil (BVO), Bisphenol A (BPA), animal and/or dairy products, gluten, or artificial additives, preservatives, flavors, or colors, and wherein the glucose tolerance test beverage composition has a variance threshold of no more than 5%. In various embodiments, the beverage composition is formulated as a soluble powder or a ready-to-drink beverage, particularly a pre-mixed 10 oz. beverage. Methods are also provided for using the beverage composition to perform one-step or two-step oral glucose tolerance tests for gestational diabetes mellitus.

### CROSS-REFERENCE TO RELATED APPLICATIONS

The present invention is a PCT International Patent Application that claims priority to U.S. Provisional Patent Application No. 63/233,025, filed on Aug. 13, 2021, the entire disclosure of which is incorporated herein by reference.

### TECHNICAL FIELD

Aspects described herein relate to glucose tolerance beverages and methods of use thereof.

### BACKGROUND

Glucose tolerance tests (also known as glucose load tests) are used to screen for diabetes mellitus and gestational diabetes mellitus. The screening entails administering a precise dose of glucose to a patient via an oral glucose beverage, and then measuring the patient's plasma blood glucose concentration at a specific time interval or intervals. For gestational diabetes mellitus screening, the American Diabetes Association suggests one of two approaches. The one-step approach utilizes a 75-gram glucose beverage, and the two-step approach utilizes a 50-gram and 100-gram glucose beverage. Determining a patient's plasma blood glucose concentration profile in response to the intake of glucose is used to identify anomalies of glucose metabolism and allow diabetes mellitus to be diagnosed.

Glucose tolerance beverages for use in glucose tolerance tests have been manufactured and sold since the 1960s. Referred to generally as “Glucola” beverages, currently available and FDA Registered glucose tolerance beverages include Azer Scientific Glucose Tolerance Test Beverages (Azer Scientific, Morgantown, PA) and Thermo Scientific™ NERL™ Trutol™ Glucose Tolerance Test Beverages (ThermoFisher Scientific, Middletown, VA). Unfortunately, currently available Glucola beverages include dextrose (D-glucose) derived from genetically modified (GMO) corn and unnecessary additives and preservatives such as brominated vegetable oil (BVO), butylated hydroxyanisole (BHA), sodium benzoate, sodium hexametaphosphate, Yellow #6, and Red #40. In addition, these traditional Glucola beverages are often manufactured in disposable plastic bottles containing bisphenol A (BPA).

Adverse reactions to Glucola beverages include nausea, vomiting, abdominal bloating, and headache, with 30% of women being tested for gestational diabetes mellitus unable to tolerate Glucola (Racusin D. A., et al. (2013). Diabetes Care, 36(10):e169-170). Women are becoming increasingly health conscious and are choosing to consume healthy, clean, and organic products, especially during pregnancy. As a result, many women request an alternative to Glucola beverages. Women's health influencers such as Food Babe, Wellness Mama, and Mama Natural have slammed traditional Glucola beverages for containing unnatural ingredients (Racusin D. A., et al. (2013). Diabetes Care, 36(10):e169-170. Furthermore, in spite of some reports that food alternatives such as jelly beans or Twizzlers may be used as a substitute for Glucola beverages (Racusin D. A., et al. (2013). Diabetes Care, 36(10):e169-170; Lamar, M. E., et al. (1999) Am. J. Obstet. Gynecol., 181(5):1154-1157) these food products are made with sucrose instead of glucose, which causes inaccurate results (one disaccharide sucrose molecule is composed of one-half glucose and one-half fructose, therefore providing only half the intended amount of glucose).

Accordingly, there is a need for safe, palatable, and effective glucose tolerance beverages that do not include unnecessary additives and preservatives.

### SUMMARY

To address the foregoing problems, in whole or in part, and/or other problems that may have been observed by persons skilled in the art, the present disclosure provides compositions and methods as described by way of example as set forth below.

A glucose tolerance test beverage composition is provided, comprising:
a) 50 g. 75 g, or 100 g dextrose from a non-GMO (Genetically Modified Organism) source;
b) organic peppermint leaf; and
c) organic or crystalized lemon;
   wherein the beverage composition is free of or substantially free of protein or DNA from GMOs, Brominated Vegetable Oil (BVO), Bisphenol A (BPA), animal and/or dairy products, gluten, or artificial additives, preservatives, flavors, or colors, and wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn. In some embodiments, the beverage composition comprises organic peppermint leaf but not organic or crystalized lemon.

A glucose tolerance test beverage composition is provided, consisting of, or consisting essentially of:
a) 50 g, 75 g, or 100 g dextrose from a non-GMO source;
b) organic peppermint leaf; and
c) organic or crystalized lemon;
   wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the beverage composition comprises organic peppermint leaf but not organic or crystalized lemon.

A method is also provided for performing a two-step oral glucose tolerance test for gestational diabetes mellitus, comprising the steps of:
a) administering a glucose tolerance beverage composition comprising 50 g dextrose to the patient;
b) obtaining a first blood glucose level measurement in the patient 60 minutes after administering the glucose tolerance beverage composition to the patient, wherein if the first blood glucose level measurement is ≥130 mg/dL, step (c) is performed;
c) obtaining a second blood glucose level measurement in the patient after fasting overnight;
d) administering a glucose tolerance beverage composition comprising 100 g dextrose to the patient; and
e) obtaining a third blood glucose level measurement in the patient, wherein a diagnosis of gestational diabetes mellitus is indicated if at least two of the following plasma glucose levels are met or exceeded:
   i) 95 mg/dL for the second blood glucose level measurement;
   ii) 180 mg/dL for the third blood glucose level measurement following 1 hour;
   iii) 155 mg/dL for the third blood glucose level measurement following 2 hours; or
   iv) 140 mg/dL for the third blood glucose level measurement following 3 hours;
      wherein the glucose tolerance beverage compositions comprise dextrose from a non-GMO source, organic peppermint leaf, and organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn. In some embodiments, the beverage composition comprises organic peppermint leaf but not organic or crystalized lemon.

A method is also provided for performing a one-step oral glucose tolerance test for gestational diabetes mellitus, comprising the steps of:
a) obtaining a fasting blood glucose level measurement in a patient who has fasted overnight;
b) administering a glucose tolerance beverage composition comprising 75 g dextrose to the patient;
c) obtaining a challenge blood glucose level measurement in the patient, wherein a diagnosis of gestational diabetes mellitus is indicated if at least one of the following plasma glucose levels are met or exceeded:
   i) 92 mg/dL for the fasting blood glucose level measurement;
   ii) 180 mg/dL for the second challenge blood glucose level measurement following 1 hour; or
   iii) 153 mg/dL for the second challenge blood glucose level measurement following 2 hours;
      wherein the glucose tolerance beverage compositions comprise dextrose from a non-GMO source, organic peppermint leaf, and organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn. In some embodiments, the beverage composition comprises organic peppermint leaf but not organic or crystalized lemon.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### DETAILED DESCRIPTION

The subject matter of the present invention now will be described more fully hereinafter, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions. Therefore, it is to be understood that the subject matter of the present invention is not to be limited to the specific embodiments disclosed and that modifications and other embodiments are intended to be included within the scope of the appended claims.

### I. Compositions

A glucose tolerance beverage composition is provided, comprising:
a) 50 g, 75 g, or 100 g dextrose from a non-GMO source;
b) organic peppermint leaf; and
c) organic or crystalized lemon;
   wherein the beverage composition is free of or substantially free of protein or DNA from Genetically Modified Organisms (GMOs), Brominated Vegetable Oil (BVO), Bisphenol A (BPA), animal and/or dairy products, gluten, or artificial additives, preservatives, flavors, or colors. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn.

A glucose tolerance beverage composition is also provided, consisting of, or consisting essentially of:
a) 50 g, 75 g, or 100 g dextrose from a non-GMO source;
b) organic peppermint leaf; and
c) organic or crystalized lemon.
   In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn.

### II. Methods

The oral glucose tolerance test is the “gold standard” for diagnosing gestational diabetes mellitus.

An oral glucose tolerance test is still or may be used in the following clinical conditions: a random blood glucose is equivocal, as part of special clinical investigation, in pregnancy (diagnosis of gestational diabetes mellitus), for experimental and epidemiological purposes, and finally to exclude or make the diagnosis of diabetes and impaired glucose tolerance.

The patient is administered 50 g, 75 g, or 100 g of glucose in a beverage composition.

A method is also provided for performing a two-step oral glucose tolerance test for gestational diabetes mellitus, comprising the steps of:
a) administering a glucose tolerance beverage composition comprising 50 g dextrose to the patient;
b) obtaining a first blood glucose level measurement in the patient 60 minutes after administering the glucose tolerance beverage composition to the patient, wherein if the first blood glucose level measurement is ≥130 mg/dL, step (c) is performed;
c) obtaining a second blood glucose level measurement in the patient after fasting overnight;
d) administering a glucose tolerance beverage composition comprising 100 g dextrose to the patient; and
e) obtaining a third blood glucose level measurement in the patient, wherein a diagnosis of gestational diabetes mellitus is indicated if at least two of the following plasma glucose levels are met or exceeded:
   i) 95 mg/dL for the second blood glucose level measurement;
   ii) 180 mg/dL for the third blood glucose level measurement following 1 hour;
   iii) 155 mg/dL for the third blood glucose level measurement following 2 hours; or
   iv) 140 mg/dL for the third blood glucose level measurement following 3 hours;
      wherein the glucose tolerance beverage compositions comprise dextrose from a non-GMO source, organic peppermint leaf, and organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn. In some embodiments, the beverage composition comprises organic peppermint leaf but not organic or crystalized lemon.

A method is also provided for performing a one-step oral glucose tolerance test for gestational diabetes mellitus, comprising the steps of:
a) obtaining a fasting blood glucose level measurement in a patient who has fasted overnight;
b) administering a glucose tolerance beverage composition comprising 75 g dextrose to the patient;
c) obtaining a challenge blood glucose level measurement in the patient, wherein a diagnosis of gestational diabetes mellitus is indicated if at least one of the following plasma glucose levels are met or exceeded:
   i) 92 mg/dL for the fasting blood glucose level measurement;
   ii) 180 mg/dL for the second challenge blood glucose level measurement following 1 hour; or
   iii) 153 mg/dL for the second challenge blood glucose level measurement following 2 hours;
      wherein the glucose tolerance beverage compositions comprise dextrose from a non-GMO source, organic peppermint leaf, and organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%. In some embodiments, the beverage composition is formulated as a soluble powder. In other embodiments, the beverage composition is formulated as a ready-to-drink beverage, particularly a pre-mixed 10 oz. beverage. In some embodiments, the dextrose from a non-GMO source is derived from tapioca (cassava root) or corn. In some embodiments, the beverage composition comprises organic peppermint leaf but not organic or crystalized lemon.

### III. Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the terms “patient” or “subject” are used interchangeably. The patient or subject treated by the presently disclosed compositions and methods is desirably a human subject, although it is to be understood that the methods described herein are effective with respect to all vertebrate species, which are intended to be included in the term “subject.” Accordingly, a “subject” can include a human subject for medical purposes (e.g., the diagnosis or treatment of an existing disease, disorder, or condition, or the prophylactic diagnosis or treatment for preventing the onset of a disease, disorder, or condition).

As used herein, the term “substantially free of” refers to less than about 0.01% or less of a substance by weight, particularly about 0.005% or less of a substance by weight, and even more particularly about 0.001% or less of a substance by weight. Alternatively, the term “substantially free of” refers to less than or equal to 20 parts per million (ppm) of a substance, more particularly to less than or equal to 10 ppm of a substance.

### EXAMPLES

### Example 1

A glucose tolerance beverage composition (The Fresh Test™) was prepared, comprising:
a) 50 g, 75 g, or 100 g non-GMO dextrose (Source: corn or tapioca cassava root);
b) organic or crystalized lemon (citric acid, lemon oil, lemon juice); and
c) organic peppermint leaf powder;
   wherein the beverage composition was free or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%.

The Fresh Test™ was formulated as a soluble powder and manufactured to food and beverage industry standards as well as Current Good Manufacturing Practice (cGMP) and other U.S. Food and Drug Administration (FDA) regulations. The product has a shelf life of 30 months and is stored between 2° and 30° C.

As described elsewhere herein, The Fresh Test™ has extra quality control and variance standards that traditional Glucola beverages do not. When manufacturing, The Fresh Test™ only allows for a 1%-5% variance threshold whereas traditional Glucola companies have a 7.5% variance threshold, as noted in their certificates of analysis. This means that The Fresh Test™ provides the most accurate dose of 50 g, 75 g, or 100 g dextrose.

### Example 2

The American College of Obstetricians and Gynecologists recommends a “one-step” or “two-step” approach for screening for gestational diabetes mellitus between 24-28 weeks of gestation in women not previously diagnosed with diabetes. These guidelines were printed in the American Diabetes Association's “Standards of Medical Care In Diabetes—2019” in Diabetes Care, 42(Suppl. 1):S1-S193. One of skill in the art may readily refer to the American Diabetes Association, the National Diabetes Data Group, the World Health Organization, the International Association of Diabetes and Pregnancy, or the American College of Obstetricians and Gynecologists for the latest guidelines and thresholds.

A two-step oral glucose tolerance test for gestational diabetes mellitus using a glucose tolerance beverage composition of Example 1 may be performed by carrying out the following steps:
a) administering a glucose tolerance beverage composition comprising 50 g dextrose to the patient;
b) obtaining a first blood glucose level measurement in the patient 60 minutes after administering the glucose tolerance beverage composition to the patient, wherein if the first blood glucose level measurement is ≥130 mg/dL, step (c) is performed;
c) obtaining a second blood glucose level measurement in the patient after fasting overnight;
d) administering a glucose tolerance beverage composition comprising 100 g dextrose to the patient; and
e) obtaining a third blood glucose level measurement in the patient, wherein a diagnosis of gestational diabetes mellitus is indicated if at least two of the following plasma glucose levels are met or exceeded:
   i) 95 mg/dL for the second blood glucose level measurement;
   ii) 180 mg/dL for the third blood glucose level measurement following 1 hour;
   iii) 155 mg/dL for the third blood glucose level measurement following 2 hours;or
   iv) 140 mg/dL for the third blood glucose level measurement following 3 hours.

### Example 3

A one-step oral glucose tolerance test for gestational diabetes mellitus using a

glucose tolerance beverage composition of Example 1 may be performed by carrying out the following steps:
a) obtaining a fasting blood glucose level measurement in a patient who has fasted overnight;
b) administering a glucose tolerance beverage composition comprising 75 g dextrose to the patient;
c) obtaining a challenge blood glucose level measurement in the patient, wherein a diagnosis of gestational diabetes mellitus is indicated if at least one of the following plasma glucose levels are met or exceeded:
   i) 92 mg/dL for the fasting blood glucose level measurement;
   ii) 180 mg/dL for the second challenge blood glucose level measurement following 1 hour; or
   iii) 153 mg/dL for the second challenge blood glucose level measurement following 2 hours.

### Example 4

An open-label comparative crossover study was conducted comparing a 50 g glucose tolerance beverage of Example 1 to a traditional 50 g Glucola drink.

Over the period of six months, the Women and Children's Clinic of Huntington Park, California, enrolled patients to participate in an open-label comparative crossover study

This study was designed to determine whether the 50 g glucose tolerance beverage formulation of Example 1 would yield equivalent levels of plasma glucose concentrations with a greater level of tolerability for patients compared to the traditional 50 g Glucola drink.

Each participant served as her own control. The inclusion criteria were: pregnant women over 18 years of age and 24-28 weeks of gestation. The exclusion criteria included: subjects under the age of 18, and subjects previously diagnosed with type 1 diabetes, type 2 diabetes, or gestational diabetes.

Case report forms were filled out for every patient. The forms contained data for glucose load test dates, what patients had to eat or drink on the day of the test, and plasma glucose test results.

All patients included in the study came in within the same week of gestation to consume both drinks but on different days.

Patients were asked their opinion of the drink and if they had any adverse reactions. The patients ranked two things—how the product tasted and how tolerable it was to drink, both on a scale of 1-5.

Patients experienced the same result when consuming the 50 g glucose tolerance beverage formulation of Example 1 as they did traditional the Glucola drink.

### Example 4

Laboratory testing was conducted on the 50 g glucose tolerance beverage of Example 1.

The product was found to be manufactured within an acceptable range of 50 g of glucose (50.00 g-50.40 g) and contain negligible amounts of fructose, sucrose, lactose, and maltose (e.g., less than 0.50 g, more particularly less than 0.27 g), as well as negligible amounts of fat, fiber, and protein.

The product was free from harmful microbiology, showing negative labs for E. Coli, Listeria, and Salmonella.

The product was also free or substantially free of protein or DNA from GMOs, BVO, BPA, animal and/or dairy products, gluten, or artificial additives, preservatives, flavors, or colors.

The product also had a variance threshold of no more than 5%.

### REFERENCE STATEMENT

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A glucose tolerance beverage composition, comprising:
a) 50 g, 75 g, or 100 g dextrose from a non-GMO (Genetically Modified Organism) source; and
b) organic peppermint leaf and/ororganic or crystalized lemon;
wherein the beverage composition is free of or substantially free of protein or DNA from GMOs, Brominated Vegetable Oil (BVO), Bisphenol A (BPA), animal and/or dairy products, gluten, or artificial additives, preservatives, flavors, or colors, and wherein the glucose tolerance test beverage composition has a variance threshold of no more than 5%.

2. The glucose tolerance beverage composition of claim 1, wherein the beverage composition is formulated as a soluble powder.

3. The glucose tolerance beverage composition of claim 1, wherein the beverage composition is formulated as a ready-to-drink beverage.

4. The glucose tolerance beverage composition of claim 3, comprising a pre-mixed 10 oz. beverage.

5. The glucose tolerance beverage composition of claim 1, wherein the dextrose from a non-GMO source is derived from tapioca (cassava root) or corn.

6. A glucose tolerance beverage composition is provided, consisting of, or consisting essentially of:
a) 50 g, 75 g, or 100 g dextrose from a non-GMO (Genetically Modified Organism) source; and
b) organic peppermint leaf and/or organic or crystalized lemon;
wherein the glucose tolerance test beverage composition has a variance threshold of no more than 5%.

7. The glucose tolerance beverage composition of claim 6, wherein the beverage composition is formulated as a soluble powder.

8. The glucose tolerance beverage composition of claim 7, wherein the beverage composition is formulated as a ready-to-drink beverage.

9. The glucose tolerance beverage composition of claim 8, comprising a pre-mixed 10 oz. beverage.

10. The glucose tolerance beverage composition of claim 6, wherein the dextrose from a non-GMO source is derived from tapioca (cassava root) or corn.

11. A method for performing a two-step oral glucose tolerance test for gestational diabetes mellitus, comprising the steps of:
a) administering a glucose tolerance beverage composition comprising 50 g dextrose to the patient;
b) obtaining a first blood glucose level measurement in the patient 60 minutes after administering the glucose tolerance beverage composition to the patient, wherein if the first blood glucose level measurement is ≥130 mg/dL, step (c) is performed;
c) obtaining a second blood glucose level measurement in the patient after fasting overnight;
d) administering a glucose tolerance beverage composition comprising 100 g dextrose to the patient; and
e) obtaining a third blood glucose level measurement in the patient, wherein a diagnosis of gestational diabetes mellitus is indicated if at least two of the following plasma glucose levels are met or exceeded:
i) 95 mg/dL for the second blood glucose level measurement;
ii) 180 mg/dL for the third blood glucose level measurement following 1 hour;
iii) 155 mg/dL for the third blood glucose level measurement following 2 hours; or
iv) 140 mg/dL for the third blood glucose level measurement following 3 hours;
wherein the glucose tolerance beverage compositions comprise dextrose from a non-GMO source, and organic peppermint leaf and/or organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%.

12. The method of claim 11, wherein the beverage composition is formulated as a soluble powder.

13. The method of claim 11, wherein the beverage composition is formulated as a ready-to-drink beverage.

14. The method of claim 13, comprising a pre-mixed 10 oz. beverage.

15. The method of claim 11, wherein the dextrose is derived from tapioca (cassava root) or corn.

16. A method for performing a one-step oral glucose tolerance test for gestational diabetes mellitus, comprising the steps of:
a) obtaining a fasting blood glucose level measurement in a patient who has fasted overnight;
b) administering a glucose tolerance beverage composition comprising 75 g dextrose to the patient;
c) obtaining a challenge blood glucose level measurement in the patient, wherein a diagnosis of gestational diabetes mellitus is indicated if at least one of the following plasma glucose levels are met or exceeded:
i) 92 mg/dL for the fasting blood glucose level measurement;
ii) 180 mg/dL for the second challenge blood glucose level measurement following 1 hour; or
iii) 153 mg/dL for the second challenge blood glucose level measurement following 2 hours;
wherein the glucose tolerance beverage compositions comprise dextrose from a non-GMO source, and organic peppermint leaf and/or organic or crystalized lemon; wherein the glucose tolerance beverage compositions are free of or substantially free of protein or DNA from GMOs, BVO, BPA, animal and/or dairy products, gluten, or artificial additives, preservatives, flavors, or colors; and wherein the glucose tolerance test beverage composition has a variance threshold of no more than 5%.

17. The method of claim 16, wherein the beverage composition is formulated as a soluble powder.

18. The method of claim 16, wherein the beverage composition is formulated as a ready-to-drink beverage.

19. The method of claim 18, comprising a pre-mixed 10 oz. beverage.

20. The method of claim 16, wherein the dextrose from a non-GMO source is derived from tapioca (cassava root) or com.
